(19) United States
(12) Patent Application Publication
(10) Pub. No.: US 2009/0048567 A1
(43) Pub. Date: Feb. 19, 2009

(54) PATCH

(76) Inventors: Quinton Van Rooyen, Windhoek (NA); Christina Van Rooyen, Windhoek (NA); Johannes Jones, Windhoek (NA); Abraham Josua Le Roux, Somerset West (ZA); Cornelius Johannes Powell, Windhoek (NA)

Correspondence Address: HAHN AND MOODLEY, LLP, P.O. BOX 52050, MINNEAPOLIS, MN, 55402, US

(21) Appl. No.: 12/093,913

(22) PCT Filed: Nov. 20, 2006
(86) PCT No.: PCT/ZA2006/000131

§ 371 Date: Nov. 6, 2008

(30) Foreign Application Priority Data
Nov. 26, 2005 (GB) 0524121.1

(51) Int. Cl. A61F 13/02

(52) U.S. Cl. 604/290; 604/306

(57) ABSTRACT

The invention provides a patch device adapted for use in the transdermal administration to a patient of a composition including one or more pharmaceutically, cosmetically, pesticidally, prophylactically, and nutritionally active substance, the patch consisting of a layered construct adapted to be adhered to the skin of a patient and defining a cavity for receiving one or more disruptable containers of the composition or components of the composition to be administered between a proximal layer and a distal layer thereof, which proximal layer is adapted in use to be located in intimate contact with the skin of the patient and which distal layer is in use disposed on the outer side thereof, wherein the proximal layer is partially permeable to that composition, so that in use the integrity of the one or more containers may be disrupted thereby causing the composition to be disposed in the cavity and permeate from there through the proximal layer to be absorbed through the skin into the body of the patient. The invention further provides a method of applying an irritant to the skin of a user.

### FIELD OF THE INVENTION

This invention relates to a skin patch or plaster suitable for use in the transdermal delivery to the human or animal body of pharmaceutical active substances or compositions containing such active substances.

### BACKGROUND TO THE INVENTION

It is well known in the field of drug delivery that certain pharmaceutical active substances may be delivered transdermally to a patient in need thereof. It is also known that many pharmaceutical active substances are not suitable as such for transdermal administration as they are not readily absorbed through the skin or cause irritation to the skin when applied directly to the skin. It is also known that the absorption of such substances through the skin of the patient may in some cases be enhanced by the use of various excipients used in the formulation of pharmaceutical preparations and in particular by the use of substances known as penetration enhancing agents. It is however further known that some of the known penetration enhancing agents and other excipients conventionally used in the formulation of pharmaceutical preparations cause irritation when applied to the skin.

Various solutions have been proposed to overcome the problem of skin irritation by compositions intended to be used for transdermal administration of pharmaceutical preparations. The recently granted U.S. Pat. No. 6,579,865 to Mak et al discloses one such attempt and refers to several others.

The recently published PCT/IB2004/052616 discloses a patch device adapted for use in the transdermal administration to a patient of a composition including, or consisting of, a selected irritating substance, the patch consisting of a layered construct adapted to be adhered to the skin of a patient and defining a depot cavity for the composition to be administered between a proximal layer and a distal layer thereof, which proximal layer is adapted in use to be located in intimate contact with the skin of the patient and which distal layer is in use disposed on the outer side thereof, the distal layer being characterised in that it is substantially impervious to the composition to be administered, and the proximal layer being characterised in that it is partially permeable to that composition, so that in use the composition may be disposed in the cavity and permeate from there through the proximal layer to be absorbed through the skin into the body of the patient, the proximal layer being further characterised in that its permeability to the irritating substance or component of the composition to be administered to the human or animal is less than the permeability of the human or animal skin, as the case may be, to such irritating substance or component.

One of the difficulties with a patch such as that disclosed in the above PCT application is that the depot cavity which is formed between the proximal and distal layers is filled by a user with one or more of a carrier substance and the active irritating substance which does not ensure the correct dosage regime. Furthermore, shelf life issues also arise as the patch is made from a porous material.

It is also known that dimethylformamide (DMF) is useful as a penetration enhancing agent. It is referred to in that context in several patents including U.S. Pat. No. 6,214,374 (Schmirler, et al.). It has also been suggested in WO97/22248US that DMF may be used as an active ingredient in combating HIV-AIDS. In WO 99/13885 it is more generally suggested that DMF may be used to affect the immune response of the body to a variety of ailments. There has however not been a suitably reliable transdermal administration device that can be used for the purpose of administering DMF at the low and sub-toxic dosages that are considered necessary to apply such proposed treatment.

One of the difficulties with applying DMF by means of conventional transdermal devices, is that it causes severe skin irritation in the form of pruritic erythema. DMF is in addition also a solvent for a large number of synthetic materials, and permeates through a number of others, both of which factors limit the choice of materials which may be used in constructing a patch suitable for use in administering DMF or DMF containing compositions to a patient-human or animal.

### OBJECT OF THE INVENTION

It is an object of the present invention to provide a patch construction which is suitable to be used in general, as a means transdermally to administer pharmaceutically, cosmetically, pesticidally, prophylactically, or nutritionally active substances and compositions which are in a fluid form, for example, for the administration of vitamin compositions, irritants, acaracides, and the like to humans and other animals.

### BRIEF DESCRIPTION OF THE INVENTION

According to the invention there is provided a patch device adapted for use in the transdermal administration to a patient of a composition including one or more pharmaceutically, cosmetically, pesticidally, prophylactically, and nutritionally active substance, the patch consisting of a layered construct adapted to be adhered to the skin of a patient and defining a cavity for receiving one or more disruptable containers of the composition or components of the composition to be administered between a proximal layer and a distal layer thereof, which proximal layer is adapted in use to be located in intimate contact with the skin of the patient and which distal layer is in use disposed on the outer side thereof, wherein the proximal layer is partially permeable to that composition, so that in use the integrity of the one or more containers may be disrupted thereby causing the composition to be disposed in the cavity and permeate from there through the proximal layer to be absorbed through the skin into the body of the patient.

The disruptable container may be impervious or substantially impervious to said composition or selected components thereof.

The disruptable container may be made of a brittle substance such as a ceramic, or of a glass composition, or any other frangible material.

The disruptable container may be an ampoule.

The ampoule may be a glass ampoule.

The use of a glass ampoule may permit an extensive shelf life for the patch as aggressive or sensitive components of the composition to be administered are stored in a glass container extending the shelf life of the patch. The use of glass for the ampoule provides for reliable release of the contents of the container into the cavity once the ampoule is broken by crushing between the fingers of a user.

The volume of the ampoule may be selected to limit the dose size of the composition for a single dosage period to a desired dose size or to below an undesirable dose size.

The proximal layer may be selected thus that its permeability to said composition is less than the permeability of the patients skin thereto.

Said composition may include one or more of an irritant, an acaracide, one or more vitamins, for example vitamin E, pain relievers, anti-inflammatory preparations, hormonal preparations and insulin.

The irritant may be dimethylformamide commonly known as DMF.

The distal layer may be characterised in that it is substantially impervious to the composition to be administered.

The layers of the patch may be produced from any suitable material or combination of materials and the chemical composition of the proximal and distal layers may be the same of different.

Their respective permeability and impervious characteristics may be achieved by virtue of their thickness of the respective layers, or by other forms of modification of the layers.

The distal layer may thus be modified by the application thereto of a thin impervious layer to render a composite layer which is impervious to the components of the composition to be applied by means of the patch.

The distal layer may be moulded to snugly receive the container or containers and to facilitate the compressive rupturing of the containers between the fingers of a user to crush the containers and release the composition therefrom.

The patch is preferably flexible to be capable of moving with the skin for the sake of comfort, and is hence preferably produced from an elastomeric material, which is most preferably a silicone material, but it is also feasible to make the patch according to the invention with a distal sheet composed of or incorporating a thin sheet of aluminium.

In the case of the composition being an irritant substance or including an irritant substance, by providing a patch from which the irritant substance is released at a rate which is lower than the rate at which the skin absorbs such substance, it has been found that the extent of irritation is substantially reduced. This is particularly the case where the irritant is DMF.

Without wishing to be bound by theory it is believed that one of the reasons why DMF leads to erythema is that the water content of perspiration released by the skin under a patch reacts with DMF to form an irritant, possibly formic acid if allowed to remain on the skin in a quantity that is greater than the quantity which the skin is capable of absorbing. By the present invention it is sought to provide a means whereby such irritants as DMF will be released at a required dose and at a rate below the rate at which skin absorption of such substance will take place, thereby ensuring that no build-up of such substances occurs on the skin. It provides a solution to the problem in that it materially reduces, if not completely eliminates, the occurrence of erythema. The invention accordingly specifically provides for a patch suitable for use in the administration of DMF to a patient and characterised in that the proximal layer of the patch has a permeability to DMF such that DMF which is, in use, located in the cavity between the layers will be released through the proximal layer at a rate below the rate at which it is absorbed through the skin of the patient to which the patch is in use applied, thereby substantially preventing build-up of DMF in direct contact with the skin of the patient.

The volume of the container is selected such that overdosing on a single patch is inhibited.

The patch may be adapted to be adhered to the skin of the patient by being provided with a peripheral edge zone of the proximal layer being provided with a pharmaceutical acceptable adhesive layer. The adhesive layer may be covered by means of a conventional peel-off cover sheet during storage.

The adhesive layer may include a skin compatible adhesive which includes a cohesive strengthening component and a least one of the following constituents: a dry-tack component, such as a polyisobutylene; a moisture-absorbent wet-tack component such as a hydrocolloid powder, and a preservative such as pectin and/or antimicrobial agent, the adhesive being characterised in that the cohesive strengthening component comprises a polysiloxane. Such an adhesive is described in WO 98/55157.

The layers of the layered patch construct may be of the same or different chemical compositions. In one preferred form of the invention the layers may be of the same chemical composition, but may be of different thickness and/or be otherwise modified to provide for the requisite degree of imperviousness of the distal layer and permeability of the proximal layer. The distal layer may thus be a composite layer including two or more layers of the same or different materials.

The layers of a patch intended to be used for the administration of dimethylformamide as such, or of a pharmaceutical preparation containing dimethylformamide as a penetration enhancing agent, may be of the same chemical composition and may be in the form of vulcanizates of silicone. In a preferred application of this form of the invention the proximal and distal layers of the patch construct may both be produced from a silicone composition that is commercially available and marketed by Wacker-Chemie GmbH of Germany under the trade name Elastosil® which composition may during the fabrication of the layers be vulcanised by means of Curing Agent E, Cl or C6 which is also obtainable from Wacker-Chemie as is known in the trade of the production of silicone articles. Curing Agent E is constituted by a 50% paste of bis-(2,4-dichlorobenzoyl)-peroxide in silicone fluid, Curing Agent Cl is Dicumyl peroxide (98%) and Curing Agent C6 is a 45% paste of 2,5-bis-(t-butylperoxy)-2,5-dimethyl-hexane in silicone rubber. In the preferred embodiment of the invention the layers are produced from the Elastosil® R401 formulation, and most preferably the grade designated as Elastosil® R401/70 by press vulcanisation or injection moulding and with the aid of Curing Agent Cl or C6.

The composition of the layer intended for use as the proximal layer in a patch for use in the administration of dimethylformamide (DMF) as such, or of a pharmaceutical preparation containing dimethylformamide as a penetration enhancing agent, is preferably produced to have a permeability to dimethylformamide of not more than 9 mgDMF/cm2/hour. Where Elastosil® R401/70 is used as the proximal layer this rate of permeability may be achieved when the layer has a thickness of 0.25 mm. The value of 9 mgDMF/cm2/hr is slightly below the typical absorption rate of DMF through a human skin. The patch may preferably be configured to have a contact surface area of the proximal layer of from 15 to 100 cm².

Typically the patch has a contact surface area of the proximal layer of from 20 to 40 cm², say 32 cm².

The composition of the layer intended for use as the distal layer in a patch for use in the administration of dimethylformamide (DMF) as such, or of a pharmaceutical preparation containing dimethylformamide as a penetration enhancing agent, is preferably produced to be substantially impervious to DMF. Where Elastosil® R401/70 is used as the distal layer this may be achieved when the layer has a sufficient thickness but such thickness may be considered undesirable as it compromises the flexibility and overall appearance of the patch.

A modified or composite distal layer may be produced by the application of a DMF impervious layer of high density polypropylene to the inner surface thereof that in use faces the proximal layer of the patch.

In this application the Elastosil® layer may typically be 0.4 mm in thickness (which as such is permeable to DMF) and a 50 micron thick high density polypropylene film is applied to the inner surface thereof with the aid of a suitable adhesive.

A polypropylene film may be located within the cavity between the disruptable container and the proximal layer. The polypropylene film may act as a shield to inhibit shards of the disrupted container from piercing the proximal layer.

The polypropylene film may float within the cavity in that it is not bonded to any portion of the proximal or distal layers.

The adhesive may comprise the silicone adhesive marketed in the transfer tape form under the codeBRD577B by PPI Adhesive Products of Waterford in Ireland.

The adhesive used for bonding the proximal and distal layers to each other, or even the DMF impervious layer, may be a silicon adhesive or an adhesive compatible with both silicone and the composition to be administered, even if said composition includes DMF. The adhesive may be Wacker Elastosil E43.

A portion of the cavity defined between the proximal and distal layers of the patch construct may in use be provided with a solid filler material to serve as a carrier for the pharmaceutically active substance or composition to be received therein once the container's integrity is ruptured. The solid filler material may be fumed silica Aerosil 200, commercially available product obtainable from Degussa Africa (Pty) Limited.

The composition mixes with the filler material after the container is ruptured and permits prolonged permeation thereof through the proximal layer.

The patch construct may further include a self-adhesive mounting layer having a surface presenting a skin adhesive which adhesive layer is typically during pre-use storage of the product covered by a peelable cover layer, which cover layer also overlies, and hence seals off, the proximal layer of the patch construct until it is exposed by peeling off the cover layer. The mounting layer may be in the form of a commercially available product conventionally used in the production of skin plasters and sold under the name Flexifix®.

The invention extends to a multi-component patch device substantially as described above, said patch device including two or more disruptable containers so that in use the integrity of the two or more containers may be disrupted thereby causing the composition to be disposed in the cavity and permeate from there through the proximal layer to be absorbed through the skin into the body of the patient.

The invention extends to a multi-dose patch device substantially as described above, said patch device including two or more disruptable containers so that in use the integrity of the containers may be disrupted one dose at a time thereby causing a single dose or a required number of doses of the composition to be disposed in the cavity and permeate from there through the proximal layer to be absorbed through the skin into the body of the patient, while any unruptured containers remain to be ruptured for future use.

### EXAMPLE OF THE INVENTION

An example of the present invention will now be described merely to illustrate the invention and without thereby limiting the scope of the invention to the exemplary embodiment.

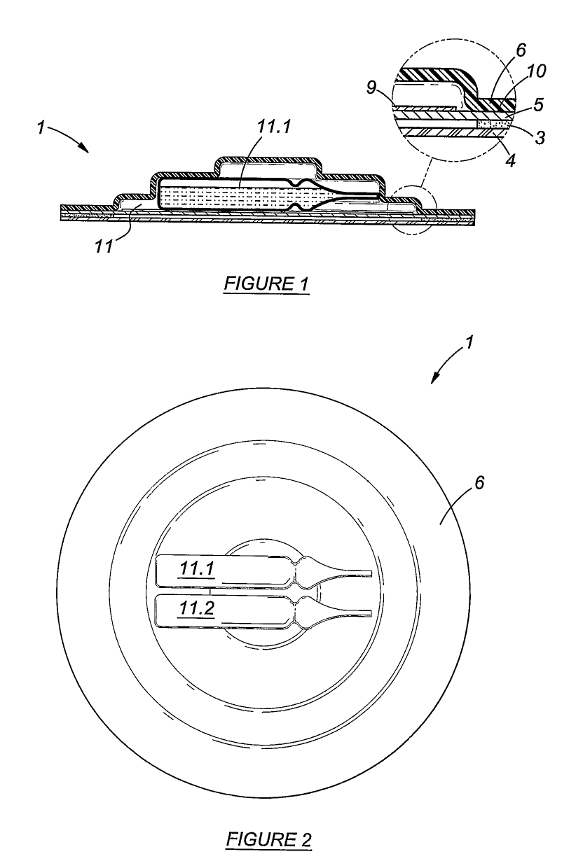

FIG. 1: In this Figure is shown a sectional view of an embodiment of a patch in accordance with the invention; and

FIG. 2: In this Figure is shown a top view of another embodiment of the patch of FIG. 1, this embodiment having two containers for components of a composition or for a composition.

It is emphasised that the drawings are not to scale, and are of a schematic nature. The dimensions of some parts of the patch may be exaggerated for illustrative purposes.

Reference will now be made to the accompanying drawings in which the same reference numerals are used to indicate the same elements.

A trans-dermal patch 1 is shown to comprise a skin adhesive coated backing layer 3 and a peelable cover 4. The peelable cover 4 may be removed to expose the proximal layer 5 of the patch. The skin adhesive coated surface of the backing layer 3 may, as pointed out above, be in the form of the commercially available product described in WO 98/55157 as a skin compatible adhesive which includes a cohesive strengthening component and a least one of the following constituents: a dry-tack component, such as a polyisobutylene; a moisture-absorbent wet-tack component such as a hydrocolloid powder, and a preservative such as pectin and/or antimicrobial agent, the adhesive being characterised in that the cohesive strengthening component comprises a polysiloxane.

Onto the adhesive coated backing layer 3 is mounted a press vulcanised or injection moulded silicone distal layer 6 of the patch according to the invention which distal layer 6 is moulded in the form of a sheet having a thickness of 0.5 mm. The distal layer is preferably press or injection moulded from a silicone composition marketed by Wacker-Chemie GmbH of Germany under the trade name Elastosil® R401/70 To have the required properties. It may be suitably coloured by the addition of a compatible dye.

Between the proximal layer 5 and the distal layer 6 is defined a cavity 11 in which, in the embodiment of FIG. 1, is located a single glass ampoule 11.1 and in the embodiment of FIG. 2, are located two glass ampoules 11.1 and 11.2 of the liquid composition to be administered. The distal layer 6 is moulded to snugly receive the container or containers 11.1 and 11.2 therein and to facilitate the compressive rupturing of the containers between the fingers of a user to crush the containers and release the composition therefrom.

A film of DMF impervious membrane in the form of a perforated high density polypropylene film 9 is located between the containers 11.1, 11.2 and the proximal layer 5, thereby protecting the proximal layer from damage by shards of the containers 11.1 and 11.2 when they are disrupted. The film 9 is placed loosely between layers 5 and 6 and floats within cavity 11.

The polypropylene film 9 does not extend right to the edge of the patch and is not captured in the adhesive between the proximal 5 and distal 6 layers of the patch (as indicated in FIG. 1). In addition the proximal 5 and distal 6 layers are bonded to each other by a silicone based adhesive.

A proximal layer of silicone produced from Elastosil® R401/70 to a thickness of 0.25 mm, which is indicated at 5 is secured in place by means of the adhesive on its peripheral surface to the distal layer 6 to form cavity 11. The construct is completed by the peelable cover 4 whereby the product as a whole is sealed during storage. The adhesive in this embodiment is Wacker Elastosil E43.

The cavity 11 defined between the proximal layer 5 and the composite distal layer 6 of the patch may for some applications be provided with a charge of fumed silica Aerosil which is capable of acting as a carrier for the substance to be administered such as for DMF containing pharmaceutical preparation which is to be released into the cavity for administration to a patient.

In use the peelable layer 4 is stripped from the construct to reveal the skin adhesive presented by the peripheral zone of the backing layer 3 and the patch is then applied to the human or animal body which is to receive treatment.

Once placed and adhered in position on the body of the patient, e.g. on the chest, upper arm, on a shoulder blade or on the thigh of the patient, the container 11.1. or more than one container 11.1 and 11.2 are ruptured releasing the composition to be administered into the cavity.

The application of a DMF containing pharmaceutical preparation by means of the patch according to the invention has been tested on patients and compared to the experience gained with similar patches of a conventional construction. Whereas patients using conventional patches with the composition in issue experienced extensive irritation and skin burns in the form of pruritic erythema, such irritation and burning sensation was substantially reduced or completed absent when the plaster of the present invention was used.

Many variations of the invention are possible without thereby departing from the spirit of the invention as disclosed herein. Thus the patch may be produced in any desired shape and may, for example, be circular in shape. The patch may thus have a fully enclosed cavity into which a breakable sachet containing the substance to be administered by means of the patch is enclosed during production of the patch and a sachet piercing member is provided on a cavity facing surface of the distal layer to provide for simple breaking of the sachet to release the contents thereof into the cavity. In use the sachet may be broken without damage to the layers of the patch itself immediately before the patch is applied to the skin.

1-42. (canceled)

43. A patch device adapted for use in the transdermal administration to a patient of a composition including one or more pharmaceutically, cosmetically, pesticidally, prophylactically, and nutritionally active substance, the patch consisting of a layered construct adapted to be adhered to the skin of a patient and defining a cavity for receiving one or more disruptable containers of the composition or components of the composition to be administered between a proximal layer and a distal layer thereof, which proximal layer is adapted in use to be located in intimate contact with the skin of the patient and which distal layer is in use disposed on the outer side thereof, wherein the proximal layer is partially permeable to that composition, so that in use the integrity of the one or more containers may be disrupted thereby causing the composition to be disposed in the cavity and permeate from there through the proximal layer to be absorbed through the skin into the body of the patient.

44. A patch device as claimed in claim 43, wherein the disruptable container is an ampoule which is impervious or substantially impervious to said composition or selected components thereof and is made of a brittle substance such as a ceramic, or of a glass composition and the volume of the ampoule is selected to limit the dose size of the composition for a single dosage period to a desired dose size or to below an undesirable dose size.

45. A patch device as claimed in claim 43, wherein the proximal layer is selected thus that its permeability to said composition is less than the permeability of the patients skin thereto.

46. A patch device as claimed in claim 43, wherein said composition includes one or more of an irritant, an acaracide, one or more vitamins, pain relievers, anti-inflammatory preparations, hormonal preparations and insulin.

47. A patch device as claimed in claim 46, wherein the irritant is dimethylformamide.

48. A patch device as claimed in claim 43, wherein the distal layer is substantially impervious to the composition to be administered and is moulded to snugly receive the container or containers and to facilitate the compressive rupturing of the containers between the fingers of a user to crush the containers and release the composition therefrom.

49. A patch device as claimed in claim 43 which includes a distal sheet composed of or incorporating a thin sheet of aluminium.

50. A patch device as claimed in claim 43, wherein a polypropylene film is located within the cavity between the disruptable container and the proximal layer, which polypropylene film acts as a shield to inhibit shards of the disrupted container from piercing the proximal layer, which polypropylene film floats within the cavity in that it is not bonded to any portion of the proximal or distal layers.

51. A patch device as claimed in claim 43, which is adapted to be adhered to the skin of the patient by being provided with a peripheral edge zone of the proximal layer being provided with a pharmaceutical acceptable adhesive layer.

52. A patch device as claimed in claim 51, wherein the adhesive layer is covered by means of a conventional peel-off cover sheet during storage.

53. A patch device as claimed in claim 51, wherein the adhesive layer includes a skin compatible adhesive which includes a cohesive strengthening component and a least one of the following constituents: a dry-tack component, such as a polyisobutylene; a moisture-absorbent wet-tack component such as a hydrocolloid powder, and a preservative such as pectin and/or antimicrobial agent, the adhesive being characterised in that the cohesive strengthening component comprises a polysiloxane.

54. A patch device as claimed in claim 43, wherein the layers of the layered patch construct are of the same or different chemical compositions.

55. A patch device as claimed in claim 43, wherein a portion of the cavity defined between the proximal and distal layers of the patch construct is in use provided with a solid filler material to serve as a carrier for the pharmaceutically active substance or composition to be received therein once the container's integrity is ruptured.

56. A patch device as claimed in claim 55, wherein the solid filler material is fumed silica.

57. A multi-component patch device as claimed in claim 56, said patch device includes two or more disruptable containers so that in use the integrity of the two or more containers may be disrupted thereby causing the composition to be disposed in the cavity and permeate from there through the proximal layer to be absorbed through the skin into the body of the patient.

58. A multi-component patch as claimed in claim 57, wherein said patch device includes two or more disruptable containers so that in use the integrity of the containers may be disrupted one dose at a time thereby causing a single dose or a required number of doses of the composition to be disposed in the cavity and permeate from there through the proximal layer to be absorbed through the skin into the body of the patient, while any unruptured containers remain to be ruptured for future use.

59. A method of applying an irritant composition to the skin of a recipient for transcutaneous absorption thereof, which method uses a patch device as claimed in claim 46, wherein the irritant is released at a rate which is lower than the rate at which the skin absorbs such substance, thereby reducing the extent of irritation of the skin.
